(19) United States
(12) Patent Application Publication
(10) Pub. No.: US 2023/0381223 A1
(43) Pub. Date: Nov. 30, 2023

(54) ORAL PYROPHOSPHATE FOR USE IN REDUCING CALCIFICATION

(71) Applicant: PYROGENYX INC., Sunnyvale, CA (US)

(72) Inventors: Donald A. McCarthy, San Francisco, CA (US); Jim W. Larrick, Sunnyvale, CA (US); Cyrus K. Becker, Pleasanton, CA (US); Meenakshi S. Venkatraman, Fremont, CA (US); Xiaoming Zhang, Sunnyvale, CA (US); Viola Pomozi, Budapest (HU); András VÁRADI, AMSTERDAM (NL)

(21) Appl. No.: 18/031,755

(22) PCT Filed: Oct. 14, 2021
(86) PCT No.: PCT/IB2021/059448

§ 371 Date: Apr. 13, 2023

Related U.S. Application Data
(60) Provisional application No. 63/091,467, filed on Oct. 14, 2020.

(51) Int. Cl. A61K 33/42; A61K 9/48; A61P 9/00; A61K 31/198

(52) U.S. Cl. CPC A61K 33/42; A61K 9/4825; A61K 9/4816; A61P 9/00; A61K 31/198

(57) ABSTRACT

The current invention relates to use of oral pyrophosphate wherein said pyrophosphate is selected from the group consisting of monoarginine pyrophosphate, monolysine pyrophosphate, dipotassium pyrophosphate, bisethanolamine pyrophosphate and bisammonium pyrophosphate for preventing and/or reducing tissue calcification, particularly soft tissue calcification, and/or diseases or disorders characterized by low plasma PPi levels, as, e.g., occurs in chronic kidney disease (CKD), end-stage renal disease (ESRD), N generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type I and II, cardiovascular disorder, calciphylaxis, calciphylaxis secondary to chronic kidney disease, calcific uremic arteriolopathy or atherosclerosis.

### FIELD OF THE INVENTION

The present invention is in the field of calcification, particularly tissue calcification, more particularly soft tissue calcification, and treatment thereof. This invention also relates to diseases or disorders the treatment of that would benefit from increasing inorganic pyrophosphate plasma levels, including such as diseases or disorders characterized by low inorganic pyrophosphate plasma levels, but also diseases or disorders characterized by normal inorganic pyrophosphate plasma levels.

### Introduction

Physiological mineralization is essential for the normal development of vertebrates. It is restricted to specific sites of the body. In mammals, biominerals predominantly consist of calcium and phosphate, together forming hydroxyapatite. In plasma and several other body fluids calcium and phosphate are present at concentrations that by far exceed their solubility constant. Vertebrates have evolved mechanisms to stabilize this supersaturated solution and to allow the regulated precipitation of calcium and phosphate only at specific bodily compartments.

Calcification (deposits of calcium phosphate) may occur in many different soft tissues in a variety of local and systemic (throughout the body) conditions. Calcium phosphate crystals have a remarkable tendency to aggregate into snowball-like clumps and are invariably associated with particular collagens. Collagens are fibrous, insoluble proteins found in the connective tissues, including skin, ligaments, and cartilage. Collagen represents about 30 percent of the total body protein.

Pyrophosphate (PPi) is a central factor in prevention of precipitation of calcium and phosphate in soft peripheral tissues. The liver is the most important source of circulatory PPi, via a pathway depending on ABCC6-mediated ATP release. Outside the hepatocytes, but still within the liver vasculature, released ATP is rapidly converted into AMP and PPi by the ectoenzyme ectonucleotide pyrophosphatase phosphodiesterase 1 (ENPP1). Pyrophosphate is a potent inhibitor of hydroxyapatite formation, and, under normal conditions, functions to inhibit soft tissue calcification, e.g., vascular calcification.

Inactivating mutations in the genes encoding the enzymes involved in PPi homeostasis result in rare hereditary calcification disorders. For example, absence of functional ABCC6 results in pseudoxanthoma elasticum (PXE), a late onset ectopic calcification disorder, with lesions found in the skin, eyes and cardiovascular system. Biallelic inactivating mutations in ENPP1 cause arterial calcification and generalized calcification of infancy (GACI), a condition that can become life-threatening shortly after birth due to massive calcification of the large and medium-sized arteries. GACI patients have virtually no PPi in their blood, which explains the severity of the disease.

As reduced PPi concentrations in the circulation underlie the ectopic calcification disorders PXE and GACI, an possible treatment for these disorders as well as other disorders characterized by reduced or too low PPi concentrations in the circulation would be PPi supplementation.

Due to the necessity to, in some instances, treat patients life-long and the short half-life of PPi, oral administration would be preferred for such a treatment. However, it has long been thought, and is therefore a reigning dogma, that PPi is ineffective when given orally (H. Fleisch, et al., Calc. Tiss. Res. 2, Suppl. (1968) 10; Francis, et al. Science (1969), 165(3899), 1264-1266; Orriss, IR., et al. Curr Opin Pharmacol. (2016) 28, 57-68). In contrast to this reigning dogma, International application WO2018052290 shows that, in animal model experiments, PPi provided to drinking water is effective when given orally.

In light of this, further or improved products, compositions, methods and uses for preventing and/or treating diseases or disorders characterized by calcification, particularly tissue calcification, particularly soft tissue calcification, or diseases or disorders the treatment of which would benefit from increasing inorganic plasma levels, including, but not limited to diseases or disorders characterized by low plasma PPi levels would be highly desirable, but are not yet readily available. In particular there is a clear need in the art for reliable, efficient and reproducible products, compositions, methods and uses that allow effective amounts of PPi to be provided to subjects in need thereof, and that have, for example, limited side-effects and/or that are easily administered to such subjects, without causing substantive discomfort. Accordingly, the technical problem underlying the present invention can been seen in the provision of such products, compositions, methods and uses for complying with any of the aforementioned needs. The technical problem is solved by the embodiments characterized in the claims and herein below.

### SUMMARY OF THE INVENTION

In a first aspect, the present disclosure relates to pyrophosphate selected from the group consisting of monoarginine pyrophosphate, monolysine pyrophosphate, dipotassium pyrophosphate, bisethanolamine pyrophosphate and bisammonium pyrophosphate for use as a medicament, and wherein said pyrophosphate is administered in oral form. In particular it was found that pyrophosphate selected from the group consisting of monoarginine pyrophosphate, monolysine pyrophosphate, dipotassium pyrophosphate, bisethanolamine pyrophosphate and bisammonium pyrophosphate is bioavailable when taken orally, in particular when it is swallowed and allowed to enter the gastro-intestinal tract beyond the oral cavity. The pyrophosphate selected from the group consisting of monoarginine pyrophosphate, monolysine pyrophosphate, dipotassium pyrophosphate, bisethanolamine pyrophosphate and bisammonium pyrophosphate is allowed to enter the gastro-intestinal tract and is absorbed, leading to an increase is systemically available pyrophosphate, as can be witnessed from increased plasma concentrations. In other words, the invention relates to the oral intake of a form of pyrophosphate that is selected from the group consisting of monoarginine pyrophosphate, monolysine pyrophosphate, dipotassium pyrophosphate, bisethanolamine pyrophosphate and bisammonium pyrophosphate. In other words, the invention relates to the systemic bioavailability of pyrophosphate by oral administration of pyrophosphate selected from the group consisting of monoarginine pyrophosphate, monolysine pyrophosphate, dipotassium pyrophosphate, bisethanolamine pyrophosphate and bisammonium pyrophosphate. It was surprisingly found that pyrophosphate that is selected from the group consisting of monoarginine pyrophosphate, monolysine pyrophosphate, dipotassium pyrophosphate, bisethanolamine pyrophosphate and bisammonium pyrophosphate is bioavailable after oral intake and the bioavailability is at least equal to but preferably greater than that of the PPi salt used in WO2018052290.

The pyrophosphate selected from the group consisting of monoarginine pyrophosphate, monolysine pyrophosphate, dipotassium pyrophosphate, bisethanolamine pyrophosphate and bisammonium pyrophosphate as an oral medicament can suitably be administered to a subject having a disease or disorder, or that is at the risk of developing such disease or disorder, that can be prevented or be treated by increasing plasma levels of inorganic pyrophosphate. Such diseases or disorder may be characterized by normal plasma levels of inorganic pyrophosphate or by low plasma levels of inorganic pyrophosphate. Also provided is pyrophosphate selected from the group consisting of monoarginine pyrophosphate, monolysine pyrophosphate, dipotassium pyrophosphate, bisethanolamine pyrophosphate and bisammonium pyrophosphate for use in preventing and/or treating diseases or disorders characterized by calcification, particularly tissue calcification, particularly soft tissue calcification, or diseases or disorders characterized by low plasma inorganic pyrophosphate (PPi) levels, wherein said pyrophosphate selected from the group consisting of monoarginine pyrophosphate, monolysine pyrophosphate, dipotassium pyrophosphate, bisethanolamine pyrophosphate and bisammonium pyrophosphate is administered in oral form, preferably in an oral form that is to be swallowed by the patient, such that the pyrophosphate selected from the group consisting of monoarginine pyrophosphate, monolysine pyrophosphate, dipotassium pyrophosphate, bisethanolamine pyrophosphate and bisammonium pyrophosphate leaves the mouth cavity and enters the gastrointestinal tract following the mouth cavity (in particular the stomach and/or intestines and/or colon).The soft tissue calcification may be vascular calcification such as arterial calcification or intimal calcification. The tissue calcification may be in a subject having ENPP1 deficiency, chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type I and II, cardiovascular disorder, calciphylaxis, calciphylaxis secondary to chronic kidney disease, calcific uremic arteriolopathy, or atherosclerosis.

In other words, there is provided for pyrophosphate selected from the group consisting of monoarginine pyrophosphate, monolysine pyrophosphate, dipotassium pyrophosphate, bisethanolamine pyrophosphate and bisammonium pyrophosphate for use in preventing and/or treating diseases or disorders wherein such diseases or disorders is characterized by calcification, particularly tissue calcification, particularly soft tissue calcification, or low plasma inorganic pyrophosphate (PPi) levels, wherein said pyrophosphate selected from the group consisting of monoarginine pyrophosphate, monolysine pyrophosphate, dipotassium pyrophosphate, bisethanolamine pyrophosphate and bisammonium pyrophosphate is administered in oral form, preferably wherein the soft tissue calcification is vascular calcification such as arterial calcification or intimal calcification, preferably wherein said disease or disorder is selected from the group consisting of chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type I and II, cardiovascular disorder, calciphylaxis, calciphylaxis secondary to chronic kidney disease, calcific uremic arteriolopathy, or atherosclerosis.

In a preferred embodiment the diseases or disorder characterized by tissue calcification is selected from the group consisting of PXE, GACI, calciphylaxis and calciphylaxis secondary to chronic kidney disease.

The pyrophosphate selected from the group consisting of monoarginine pyrophosphate, monolysine pyrophosphate, dipotassium pyrophosphate, bisethanolamine pyrophosphate and bisammonium pyrophosphate may advantageously be administered to a human subject. The pyrophosphate selected from the group consisting of monoarginine pyrophosphate, monolysine pyrophosphate, dipotassium pyrophosphate, bisethanolamine pyrophosphate and bisammonium pyrophosphate may be administered daily. The daily dose may be between 10-1000 mg of the pyrophosphate according to the invention per kilogram bodyweight.

It is understood that an oral form may be a solution such as water comprising the pyrophosphate in accordance with the invention as shown in the examples, or may be in the form of a capsule, such as a gelatin capsule, comprising the pyrophosphate in accordance with the invention. As shown in the example section, capsules may be useful for oral administration as it may avoid any unpleasant taste that can be associated with the pyrophosphates in accordance with the invention. Moreover, capsules that provide for release of the pyrophosphate from the capsule in the stomach, such as gelatin capsules or the like, may be preferred. Highly surprisingly, the use of gelatin capsules provided for a highly improved adsorption of pyrophosphate. Without being bound by theory, release in the stomach may provide for more effective absorption because at the low pH the pyrophosphate anion carries less negative charge which may allow for more efficient (passive) diffusion through the organ barrier of the gastrointestinal tract. Hence, in a preferred embodiment, a capsule or other form is selected which allows for release of the pyrophosphate in the stomach. It is highly preferred to use sodium-free pyrophosphate forms such as shown in the examples herein as the pyrophosphate to be comprised in the capsule or other form selected for release in the stomach. Such release is preferably within at most minutes of reaching the stomach. Highly preferably, gelatin capsules that allow for such fast release in the stomach are used comprising a pyrophosphate in accordance with the invention. It may also be contemplated to use such capsules, for any further suitable pyrophosphate salts.

In one embodiment, a pyrophosphate is provided, comprised in a form which releases the inorganic pyrophosphate in the stomach. It is understood that such a form may be a tablet or capsule or the like which allows for oral administration i.e. swallowing. It is also understood that the form preferably allows for release in the stomach within minutes of administration. In one embodiment, a pyrophosphate is provided as disodium pyrophosphate comprised in a form for release of the inorganic pyrophosphate in the stomach. Preferably, in one embodiment, a pyrophosphate is provided as dipotassium pyrophosphate comprised in a form for release of the pyrophosphate in the stomach. Preferably, in another embodiment, a pyrophosphate is provided, selected from the group consisting of monoarginine pyrophosphate, monolysine pyrophosphate, dipotassium pyrophosphate, bisethanolamine pyrophosphate and bisammonium pyrophosphate, comprised in a form for release of the pyrophosphate in the stomach. In another embodiment, a pyrophosphate is provided, selected from the group consisting of monoarginine pyrophosphate, monolysine pyrophosphate, dipotassium pyrophosphate, bisethanolamine pyrophosphate and bisammonium pyrophosphate, comprised in a form for release of the pyrophosphate in the stomach. In yet another further embodiment, a pyrophosphate is provided selected from monoarginine pyrophosphate and monolysine pyrophosphate, comprised in a form for release of the pyrophosphate in the stomach.

Hence, in one embodiment, a pyrophosphate is provided, comprised in a capsule which releases the pyrophosphate in the stomach, such as a gelatin capsule. In one embodiment, a pyrophosphate is provided as disodium pyrophosphate comprised in a capsule for release of the pyrophosphate in the stomach, such as preferably a gelatin capsule. Preferably, in one embodiment, a pyrophosphate is provided as dipotassium pyrophosphate comprised in a capsule for release of the pyrophosphate in the stomach, such as preferably a gelatin capsule. Preferably, in another embodiment, a pyrophosphate is provided, selected from the group consisting of monoarginine pyrophosphate, monolysine pyrophosphate, dipotassium pyrophosphate, bisethanolamine pyrophosphate and bisammonium pyrophosphate, comprised in a capsule for release of the pyrophosphate in the stomach, such as preferably a gelatin capsule. In another embodiment, a pyrophosphate is provided, selected from the group consisting of monoarginine pyrophosphate, monolysine pyrophosphate, dipotassium pyrophosphate, bisethanolamine pyrophosphate and bisammonium pyrophosphate, comprised in a capsule for releases of the pyrophosphate in the stomach, such as preferably a gelatin capsule. In yet another further embodiment, a pyrophosphate is provided selected from monoarginine pyrophosphate and monolysine pyrophosphate, comprised in a capsule for release of the pyrophosphate in the stomach, such as preferably a gelatin capsule.

In another embodiment, a pyrophosphate is provided comprised in a gelatin capsule for release of the pyrophosphate in the stomach. In another embodiment, a pyrophosphate in accordance with the invention is provided comprised in a gelatin capsule for release of the pyrophosphate in the stomach. In one embodiment, a pyrophosphate is provided as disodium pyrophosphate comprised in a gelatin capsule for release of the pyrophosphate in the stomach. Preferably, in one embodiment, a pyrophosphate is provided as dipotassium pyrophosphate comprised in a gelatin capsule for release of the pyrophosphate in the stomach. Preferably, in another embodiment, a pyrophosphate is provided, selected from the group consisting of monoarginine pyrophosphate, monolysine pyrophosphate, dipotassium pyrophosphate, bisethanolamine pyrophosphate and bisammonium pyrophosphate, comprised in a gelatin capsule for release of the pyrophosphate in the stomach. In another embodiment, a pyrophosphate is provided, selected from the group consisting of monoarginine pyrophosphate, monolysine pyrophosphate, dipotassium pyrophosphate, bisethanolamine pyrophosphate and bisammonium pyrophosphate, comprised in a gelatin capsule for release of the pyrophosphate in the stomach. In yet another further embodiment, a pyrophosphate is provided selected from monoarginine pyrophosphate and monolysine pyrophosphate, comprised in a gelatin capsule for release of the pyrophosphate in the stomach.

In a second aspect, the present disclosure provides a method for preventing and/or reducing calcification, particularly tissue calcification, particularly soft tissue, calcification, and/or diseases or disorders characterized by low plasma PPi levels comprising the step of: administering to a subject in need thereof a therapeutically effective amount of pyrophosphate selected from the group consisting of monoarginine pyrophosphate, monolysine pyrophosphate, dipotassium pyrophosphate, bisethanolamine pyrophosphate and bisammonium pyrophosphate, wherein said pyrophosphate selected from the group consisting of monoarginine pyrophosphate, monolysine pyrophosphate, dipotassium pyrophosphate, bisethanolamine pyrophosphate and bisammonium pyrophosphate is administered in oral form.

The soft tissue calcification may be vascular calcification such as arterial calcification or intimal calcification. The pyrophosphate selected from the group consisting of monoarginine pyrophosphate, monolysine pyrophosphate, dipotassium pyrophosphate, bisethanolamine pyrophosphate and bisammonium pyrophosphate may be sufficient to achieve a transient increase in plasma PPi level in the subject. The transient increase in plasma PPi level may be characterized by a PPi level that is at least about 40% of the plasma PPi level in a healthy subject. The transient increase in plasma PPi level may be maintained for at least about 15 minutes, at least 30 minutes, at least 60 minutes, at least 120 minutes, or longer. The subject may have a disease or disorder characterized by low plasma PPi levels, e.g., chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type I and II, cardiovascular disorder, calciphylaxis, calciphylaxis secondary to chronic kidney disease, calcific uremic arteriolopathy or atherosclerosis. In a preferred embodiment the diseases or disorder characterized by tissue calcification is selected from the group consisting of PXE, GACI, calciphylaxis and calciphylaxis secondary to chronic kidney disease.

In an embodiment, the subject has GACI or PXE. A daily dose of pyrophosphate selected from the group consisting of monoarginine pyrophosphate, monolysine pyrophosphate, dipotassium pyrophosphate, bisethanolamine pyrophosphate and bisammonium pyrophosphate may be between 10-1000 mg per kilogram bodyweight.

### Detailed Description of the Invention

### BRIEF DESCRIPTION OF THE DRAWINGS

Embodiments of the invention are further described hereinafter i.a. with reference to the accompanying drawings.

FIG. 1: Oral uptake of pyrophosphate in humans delivering different forms and formulations. Plasma PPᵢ levels were determined by the luminescent method. Error bars represent SD on panels (A), (B) and (C), while mean and SEM are displayed on (D). Panel A: oral delivery of Na₄P₂O₇-water solution, 40 mg/kg pyrophosphate, n=10. Panel B: oral delivery of Na₂H₂P₂O₇ in gelatin capsule formula, 39 mg/kg pyrophosphate, n=9. Panel C: oral delivery of Na₂H₂P₂O₇ in cellulose capsule formula, 39 mg/kg pyrophosphate, n=7. Panel D: comparison of uptake curves presented on Panels a, b and c, n=10, 9 and 7, respectively.

FIG. 2: Absorption of orally delivered pyrophosphate in PXE patients. Plasma PPᵢ levels were determined by the luminescent method. Error bars represent SD on panel A and C, and SEM on panel B. Panel A: oral uptake of Na₂H₂P₂O₇ in gelatin capsule formula by PXE patients, 39 mg/kg pyrophosphate, n=9. Panel B: comparison of uptake curves of healthy volunteers and PXE patients taking 39 mg/kg Na₂H₂P₂O₇ in gelatin capsules, n=9. Panel C: baseline plasma pyrophosphate levels of healthy volunteers and PXE patients involved in the study (n=9, p-value calculated by Mann-Whitney U test).

FIG. 3: Chemical structure of pyrophosphate compounds studied.

FIG. 4: Absorption of sodium-free pyrophosphate compounds and Na₂H₂P₂O₇ in mice. Plasma bioavailability of PPi after administration of pyrophosphate (PPi) selected from the group consisting of monoarginine pyrophosphate, monolysine pyrophosphate, dipotassium pyrophosphate, bisethanolamine pyrophosphate and bisammonium pyrophosphate. Plasma PPi levels of mice after dosing with various pyrophosphate compounds via gastric gavage. Dose was 39 mg/kg pyrophosphate in each case. Mean ±SEM are displayed, n3 at each data point.

FIG. 5: Absorption of sodium-free pyrophosphate compounds and Na₂H₂P₂O₇ in mice. Plasma PPi levels after administration of monolysine-H2PP1, monoarginine-H2PP1 and K2H2PPi. Plasma PPi levels of mice after dosing with various pyrophosphate compounds via gastric gavage. Dose was 39 mg/kg pyrophosphate in each case. Mean ±SEM are displayed, n≥3 at each data point.

FIG. 6: Absorption of sodium-free pyrophosphate compounds and Na₂H₂P₂O₇ in mice. Plasma PPi levels of mice after dosing with various pyrophosphate compounds via gastric gavage. Dose was 39 mg/kg pyrophosphate in each case. Mean±SEM are displayed, n≥3 at each data point.

FIG. 7: The potassium pyrophosphate K₂H₂P₂O₇ shows similar absorption characteristics in humans as Na₂H₂P₂O₇, and plasma inorganic phosphate remains in the normal range. The same individuals were involved in the absorption studies executed with the two different pyrophosphate forms. This provides the best basis of comparison. Panel A: oral uptake of K₂H₂P₂O₇ in gelatin capsule formula by healthy volunteers, 39 mg/kg pyrophosphate. Mean ±SD values are displayed, n=6. Panel B: Plasma inorganic phosphate (Pᵢ) levels of volunteers after dosing with K₂H₂P₂O₇ remain within the normal range of 0.81-1.45 mmol/L, indicated by shaded area. Mean±SD values are plotted, n=6. Panel C: Individual plasma PPᵢ (pyrophosphate) curves to compare the absorption in the same healthy volunteers from K₂H₂P₂O₇ (solid line) versus Na₂H₂P₂O₇ (dashed line). Mean ±SD values are displayed.

FIG. 8: K₂H₂P₂O₇ and Na₂H₂P₂O₇ inhibit dystrophic cardiac calcification of Abcc6^{-/-} mice when given orally. Total Ca²⁺-content of the heart tissue was measured; mean±SEM are graphed, p-values indicated were calculated by Mann-Whitney U test, animal numbers in each treatment group (n) are displayed on the figure.

### DEFINITIONS

The section headings used herein are for organizational purposes only and are not to be construed as limiting the subject matter described.

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the term “subject” includes both mammals and non-mammals. Examples of mammals include, but are not limited to, humans, chimpanzees, apes, monkeys, cattle, horses, sheep, goats, swine, rabbits, dogs, cats, rats, mice, guinea pigs, and so on. Examples of non-mammals include, without limitation, birds, fish, and the like.

As used herein, the term “therapeutically effective amount” refers to a non-toxic amount of pyrophosphate selected from the group consisting of monoarginine pyrophosphate, monolysine pyrophosphate, dipotassium pyrophosphate, bisethanolamine pyrophosphate and bisammonium pyrophosphate that is sufficient to result in improved treatment or healing of a disease or disorder, or a decrease in the rate of advancement of a disease or disorder.

The term “soft tissue” as used herein refers to the tissues that connect, support, or surround other structures and organs of the body, not being hard tissue such as bone. Soft tissue includes tendons, ligaments, fascia, skin, fibrous tissues, fat, and synovial membranes (which are connective tissue), and muscles, nerves and blood vessels (which are not connective tissue).

The term “about” as used herein is meant to denote variations of ±20% or ±10%, or ±5% or ±1% from the specified value, as such variations are still suitable to perform the methods taught herein.

The term “treating” as used herein refers to the administration of PPi in the form of pyrophosphate selected from the group consisting of monoarginine pyrophosphate, monolysine pyrophosphate, dipotassium pyrophosphate, bisethanolamine pyrophosphate and bisammonium pyrophosphate to a subject who has a diseases or disorder that can be prevented or be treated by increasing plasma inorganic pyrophosphate levels, for example but not limited to, of a subject who has a disease or disorder characterized by low PPi levels in the blood (plasma), or other progressive disorder characterized by the accumulation of deposits of calcium and other minerals, with the purpose of curing, healing, alleviating, relieving, altering, remedying, ameliorating, preventing, improving, or affecting the disease or disorder. The term “treating” refers to any indicia of success in the treatment or amelioration of an injury, pathology or condition, including any objective or subjective parameter such as abatement; remission; diminishing of symptoms; increasing tolerability of the injury, pathology or condition; slowing progression of the injury, pathology or condition; slowing the rate of degeneration or decline; or improving the subject's physical or mental well-being. Treatment may be therapeutic or prophylactic.

The term “preventing and/or reducing” as used herein refers to the prevention of calcification, the prevention of further calcification in (soft) tissues that already contain some degree of calcification as well as (partial) reversal of calcification already formed.

### DETAILED DESCRIPTION

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

As embodied and broadly described herein, the present invention is directed to the surprising finding that pyrophosphate selected from the group consisting of monoarginine pyrophosphate (e.g. monoarginine-H2PPi), monolysine pyrophosphate (e.g. monolysine-H2PPi), dipotassium pyrophosphate (e.g. K2H2PPi), bisethanolamine pyrophosphate (e.g. bisethanolamine-H2PP1) and bisammonium pyrophosphate (e.g. (NH4)2-H2PPi) when administered in oral form, and preferably when swallowed, or directly administered in the gastrointestinal tract beyond the oral cavity, has good and/or high (systemic) bioavailability and efficiently increases plasma PPi in (human) subjects, for example in comparison to the PPi as disclosed in WO2018052290. It was found that, in comparison to other (salt) forms of pyrophosphate, oral pyrophosphate selected from the group consisting of monoarginine pyrophosphate, monolysine pyrophosphate, dipotassium pyrophosphate, bisethanolamine pyrophosphate and bisammonium pyrophosphate at a substantial lower dose (expressed as PPi equivalent) may achieve comparable or even higher plasma levels of PPi in subjects. As consequence thereof sodium intake may be reduced relative to other (salt) forms of pyrophosphate. In other words, higher concentrations of plasma PPi can be obtained when equimolar amounts of pyrophosphate are administered as pyrophosphate selected from the group consisting of monoarginine pyrophosphate, monolysine pyrophosphate, dipotassium pyrophosphate, bisethanolamine pyrophosphate and bisammonium pyrophosphate in comparison to other forms of pyrophosphate. Preferably the pyrophosphate is one or more selected from the group consisting of monoarginine pyrophosphate, monolysine pyrophosphate, dipotassium pyrophosphate, bisethanolamine pyrophosphate and bisammonium pyrophosphate. Preferably the pyrophosphate is monoarginine pyrophosphate. Preferably the pyrophosphate is monolysine pyrophosphate. Preferably the pyrophosphate is dipotassium pyrophosphate. Preferably the pyrophosphate is bisethanolamine pyrophosphate. Preferably the pyrophosphate is dipotassium pyrophosphate. Pyrophosphates including monoarginine pyrophosphate, bisethanolamine pyrophosphate, dipotassium pyrophosphate, dipotassium pyrophosphate can be obtained from the prior art (see for example, Pragasam et al. Optical Materials (Amsterdam, Netherlands) (2006), 29(2-3), 173-179. D01:10.1016/j.optmat.2005.08.018, or Averbuch-Pouchot et al. European Journal of Solid State and Inorganic Chemistry (1992), 29(3), 411-18, or Yamaguchi, Hachiro Nippon Kagaku Kaishi (1978), (6), 850-3.).

Improvement of plasma PPi concentration can be achieved with a lower dose pyrophosphate selected from the group consisting of monoarginine pyrophosphate, monolysine pyrophosphate, dipotassium pyrophosphate, bisethanolamine pyrophosphate and bisammonium pyrophosphate, reducing potential side-effects/adverse effects and/or discomfort to the subject (including lower rate of gastrointestinal tract related complaints: nausea, vomiting, diarrhea, and gastric dumping) or allowing to increase total dose without increase in such undesired effects, in comparison to other forms of pyrophosphate. Common mentioned side effects include GI complaints such as nausea, vomiting, diarrhea, gastric dumping.

### Methods of Treatment

The present invention relates to use of orally administered pyrophosphate selected from the group consisting of monoarginine pyrophosphate, monolysine pyrophosphate, dipotassium pyrophosphate, bisethanolamine pyrophosphate and bisammonium pyrophosphate for treatment of calcification, for example tissue calcification, particularly in soft tissue calcification, e.g., vascular calcification. The reigning, over 50 year old, dogma has been that pyrophosphate has to be injected as it is ineffective orally because of hydrolytic destruction within the gut (Orriss et al. 2016, supra). In contrast to this reigning dogma, International application WO2018052290 shows that, in animal model experiments, PPi provided to drinking water is effective when given orally and can increase plasma PPi.

It has now surprisingly be found that oral pyrophosphate selected from the group consisting of monoarginine pyrophosphate, monolysine pyrophosphate, dipotassium pyrophosphate, bisethanolamine pyrophosphate and bisammonium pyrophosphate has high bioavailability and that oral pyrophosphate selected from the group consisting of monoarginine pyrophosphate, monolysine pyrophosphate, dipotassium pyrophosphate, bisethanolamine pyrophosphate and bisammonium pyrophosphate is highly effective in increasing plasma concentration of PPi, for example in comparison to other forms of pyrophosphate. Oral pyrophosphate selected from the group consisting of monoarginine pyrophosphate, monolysine pyrophosphate, dipotassium pyrophosphate, bisethanolamine pyrophosphate and bisammonium pyrophosphate is thus effective in increasing plasma PPi concentration and can attenuate calcification in subjects, including subjects with PXE. Hence, the present inventors have proven that orally administered pyrophosphate selected from the group consisting of monoarginine pyrophosphate, monolysine pyrophosphate, dipotassium pyrophosphate, bisethanolamine pyrophosphate and bisammonium pyrophosphate can reach the blood circulation in subjects and may counteract soft tissue calcification, making long-term treatment of soft tissue calcification disorders and diseases, some of which are hereditary and require life-long treatment, feasible. In other words, oral pyrophosphate selected from the group consisting of monoarginine pyrophosphate, monolysine pyrophosphate, dipotassium pyrophosphate, bisethanolamine pyrophosphate and bisammonium pyrophosphate may be used to treat subjects that would benefit from increasing plasma inorganic pyrophosphate level. This may be subjects characterized by low plasma PPi levels, but may also be subjects that are characterized by normal plasma PPi levels, but that would benefit from increased plasma levels PPi, for example in order to prevent urinary or salivary stone formation, or to treat urinary or salivary stone formation. In other words, the oral pyrophosphate selected from the group consisting of monoarginine pyrophosphate, monolysine pyrophosphate, dipotassium pyrophosphate, bisethanolamine pyrophosphate and bisammonium pyrophosphate provided by the current invention may suitable be used in the prevention or treatment of undesired calcification processes in the human body.

In an aspect, the present invention provides the use of pyrophosphate selected from the group consisting of monoarginine pyrophosphate, monolysine pyrophosphate, dipotassium pyrophosphate, bisethanolamine pyrophosphate and bisammonium pyrophosphate, wherein the pyrophosphate is to be administered in oral form, for use as a medicament, in particular for preventing and/or treating diseases or disorders characterized by calcification, particularly tissue calcification, particularly soft tissue calcification, and/or diseases or disorders characterized by low plasma PPi levels. Thus, the present invention provides the use of pyrophosphate selected from the group consisting of monoarginine pyrophosphate, monolysine pyrophosphate, dipotassium pyrophosphate, bisethanolamine pyrophosphate and bisammonium pyrophosphate for treating diseases or disorders characterized by calcification, particularly tissue calcification, particularly soft tissue calcification, wherein said pyrophosphate selected from the group consisting of monoarginine pyrophosphate, monolysine pyrophosphate, dipotassium pyrophosphate, bisethanolamine pyrophosphate and bisammonium pyrophosphate is administered orally, or in oral form, or is administered to the gastro-intestinal tract. The invention also provides a method for reducing calcification, in particular tissue calcification, particularly soft tissue calcification, comprising the step of administering to a subject in need thereof pyrophosphate selected from the group consisting of monoarginine pyrophosphate, monolysine pyrophosphate, dipotassium pyrophosphate, bisethanolamine pyrophosphate and bisammonium pyrophosphate, wherein said pyrophosphate selected from the group consisting of monoarginine pyrophosphate, monolysine pyrophosphate, dipotassium pyrophosphate, bisethanolamine pyrophosphate and bisammonium pyrophosphate is administered in oral form. In particular the invention also provides a method for reducing calcification, in particular tissue calcification, particularly soft tissue calcification, wherein said calcification, in particular tissue calcification, particularly soft tissue calcification is systemic, and in a preferred embodiment not in the mouth or oral cavity.

The subject to be treated may be a human patient exhibiting low (plasma) levels of pyrophosphate, suffering from a disease or disorder associated with low levels of pyrophosphate, or suffering from a progressive disorder characterized by the accumulation of deposits of calcium and other minerals (mineralization) in, for example elastic fibers (calcification). Calcification happens when calcium builds up in body tissue, blood vessels, or organs. Urinary or salivary stone formation is also to be understood as a form of calcification within the context of the current disclosure. This buildup can harden and disrupt body's normal processes. Mineralization may occur at the heart, arteries, blood vessels, kidney, spine ligaments, skin, eyes, or the digestive tract. The subject may be of any age and gender, and may have low plasma PPi, although the subject may also have normal plasma PPi levels (but would benefit from increased plasma PPi levels). Low plasma PPi may be caused by, for example, congenital deficiencies as taught herein above or others known to result in low plasma PPi levels. Low plasma PPi is also frequently seen in subjects with chronic kidney disease, end-stage renal disease/failure, diabetes mellitus and other conditions. Accordingly, the subject in need of therapy may have ENPP1 deficiency, chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type I and II, cardiovascular disorder, calciphylaxis, calciphylaxis secondary to chronic kidney disease, calcific uremic arteriolopathy or atherosclerosis. In a preferred embodiment the diseases or disorder characterized by tissue calcification is selected from the group consisting of PXE, GACI, calciphylaxis and calciphylaxis secondary to chronic kidney disease.

Other conditions that can be treated or prevented include urinary or salivary stone (formation) or any other type of undesired calcification in such subject.

The subject is preferably a human, but may also be any other suitable mammal or non-mammal.

Diseases or disorders characterized by tissue calcification, particularly soft tissue calcification, include, but are not limited to, generalized arterial calcification of infancy (GACI), pseudoxanthoma elasticum (PXE), Arterial Calcification Due to Deficiency of CD73 (ACDC), vascular calcification in chronic kidney disease (VCCKD), insulin resistance, hypophosphatemic rickets, ossification of the posterior longitudinal ligament of the spine, myocardial ischemia, joint calcification, heterotropic ossification of traumatized muscle, and angioid streaks. Also treatment of conditions that can be improved by reducing and/or eliminating one or more calcification structures and/or preventing calcification structures from forming in a subject, are within the scope of the present invention. Such conditions include, without limitation, Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, and tumoral calcinosis.

Generally, the dose of pyrophosphate selected from the group consisting of monoarginine pyrophosphate, monolysine pyrophosphate, dipotassium pyrophosphate, bisethanolamine pyrophosphate and bisammonium pyrophosphate administered to a subject in need thereof will vary depending upon age, health and weight of the subject, frequency of treatment, and the like. For example, a dose of pyrophosphate selected from the group consisting of monoarginine pyrophosphate, monolysine pyrophosphate, dipotassium pyrophosphate, bisethanolamine pyrophosphate and bisammonium pyrophosphate may be between about 0.1 mg per kg of body weight and about 1 g per kg of body weight, e.g., between about 0.5 mg per kg of body weight and about 500 mg per kg of body weight, or between about 1 mg per kg of body weight and about 300 mg per kg of body weight, or between about 10 mg per kg of body weight and about 200 mg per kg of body weight, or between about 20 mg per kg of body weight and about 150 mg per kg of body weight. Precise dose and frequency of administration can be determined by a physician skilled in the art.

In a preferred embodiment the pyrophosphate selected from the group consisting of monoarginine pyrophosphate, monolysine pyrophosphate, dipotassium pyrophosphate, bisethanolamine pyrophosphate and bisammonium pyrophosphate is provided daily. In a preferred embodiment the dosage per day is between 1-1000 mg pyrophosphate selected from the group consisting of monoarginine pyrophosphate, monolysine pyrophosphate, dipotassium pyrophosphate, bisethanolamine pyrophosphate and bisammonium pyrophosphate per kg of body weight, for example between 10-300 mg per kg of body weight, 20-200 mg per kg of body weight or between 30-100 mg per kg of body weight, for example about 50 mg per kg of body weight. It is understood that, the dosage of mg pyrophosphate per kg of body weight, refers to the amount of mg pyrophosphate per kg of body weight not taking into account the molar mass of the counter-ion, such as potassium or lysine, such as described in the examples. Hence, in another preferred embodiment, the dosage per day is about 39 mg pyrophosphate selected from the group consisting of monoarginine pyrophosphate, monolysine pyrophosphate, dipotassium pyrophosphate, bisethanolamine pyrophosphate and bisammonium pyrophosphate per kg of body weight.

The skilled physician will readily appreciate that certain factors may influence the dose or dosage required to effectively treat a subject, including, but not limited to, the severity of the disease, previous treatments, the general health and/or age of the subject, and other diseases present in the subject.

The amount of pyrophosphate selected from the group consisting of monoarginine pyrophosphate, monolysine pyrophosphate, dipotassium pyrophosphate, bisethanolamine pyrophosphate and bisammonium pyrophosphate may be in the form of a unit-dosage comprising all of the therapeutically effective amount, or may be contained in multiple dosage forms. The pyrophosphate selected from the group consisting of monoarginine pyrophosphate, monolysine pyrophosphate, dipotassium pyrophosphate, bisethanolamine pyrophosphate and bisammonium pyrophosphate may be administered once daily, twice daily, or 3, 4, ,6 , 7 ,8, 9, or 10 times daily, or the like. It will be appreciated that the effective dosage of pyrophosphate selected from the group consisting of monoarginine pyrophosphate, monolysine pyrophosphate, dipotassium pyrophosphate, bisethanolamine pyrophosphate and bisammonium pyrophosphate used for the treatment taught herein may increase or decrease over the course of the treatment.

The oral pyrophosphate selected from the group consisting of monoarginine pyrophosphate, monolysine pyrophosphate, dipotassium pyrophosphate, bisethanolamine pyrophosphate and bisammonium pyrophosphate may be administered to a subject for a period of time determined by a skilled physician. In one embodiment, e.g., for certain hereditary calcification disorders, the period of time will be the remainder of the subject's life span.

In one embodiment, the subject is an infant. The subject may be between 1 month and 24 months in age, less than 1 year of age, less than 2 years of age, less than 3 years of age, less than 4 years of age, less than 5 years of age, or less than 6 years of age.

In an embodiment, the level of blood/plasma PPi in a subject prior to treatment is less than about 80%, such as less than about 70%, less than about 60%, less than about 50%, less than about 40%, less than about 30%, less than about 25%, less than about 20%, less than about 15%, less than about 10%, less than about 5%, less than about 3%, less than about 2% or less than about 1% of normal levels of PPi observed in a healthy human subject. In an embodiment, a subject shows no measurable level of blood/plasma PPi prior to treatment.

In an embodiment, the pyrophosphate selected from the group consisting of monoarginine pyrophosphate, monolysine pyrophosphate, dipotassium pyrophosphate, bisethanolamine pyrophosphate and bisammonium pyrophosphate may be administered in conjunction with a pharmaceutically acceptable carrier, diluent, or excipient. As according to the invention the pyrophosphate selected from the group consisting of monoarginine pyrophosphate, monolysine pyrophosphate, dipotassium pyrophosphate, bisethanolamine pyrophosphate and bisammonium pyrophosphate is administered orally, it may be presented in any form suitable for such administration, e.g. in the form of tablets, capsules, powders, syrups or solutions. In a preferred embodiment, the oral pyrophosphate selected from the group consisting of monoarginine pyrophosphate, monolysine pyrophosphate, dipotassium pyrophosphate, bisethanolamine pyrophosphate and bisammonium pyrophosphate is to be swallowed by the patient or is administered to the gastrointestinal tract beyond/after the oral cavity. In one preferred embodiment, the pyrophosphate selected from the group consisting of monoarginine pyrophosphate, monolysine pyrophosphate, dipotassium pyrophosphate, bisethanolamine pyrophosphate and bisammonium pyrophosphate is administered in the form of a solid pharmaceutical entity, suitably as a tablet or a capsule. In one preferred embodiment, the pyrophosphate selected from the group consisting of monoarginine pyrophosphate, monolysine pyrophosphate, dipotassium pyrophosphate, bisethanolamine pyrophosphate and bisammonium pyrophosphate is administered in the form wherein the pyrophosphate selected from the group consisting of monoarginine pyrophosphate, monolysine pyrophosphate, dipotassium pyrophosphate, bisethanolamine pyrophosphate and bisammonium pyrophosphate is not in a solution. Methods for the preparation of solid pharmaceutical compositions or preparations are well known in the art. Thus, tablets may be prepared by mixing the active ingredient with conventional adjuvants, fillers and diluents and subsequently compressing the mixture in a suitable tableting machine. Examples of adjuvants, fillers and diluents comprise cornstarch, lactose, talcum, magnesium stearate, gelatin, gums, and the like. Typical fillers are selected from lactose, mannitol, sorbitol, cellulose and microcrystalline cellulose. Any other adjuvant or additive such as colorings, aroma, preservatives, etc, may also be used provided that they are compatible with the active ingredient. In an embodiment, the oral pyrophosphate selected from the group consisting of monoarginine pyrophosphate, monolysine pyrophosphate, dipotassium pyrophosphate, bisethanolamine pyrophosphate and bisammonium pyrophosphate is in the form of an extended release, slow release or delayed release formulation.

In an embodiment, the pyrophosphate selected from the group consisting of monoarginine pyrophosphate, monolysine pyrophosphate, dipotassium pyrophosphate, bisethanolamine pyrophosphate and bisammonium pyrophosphate may be included in a food or food supplement product, e.g., a sweet or chewing gum, or the like.

In an embodiment, the pyrophosphate selected from the group consisting of monoarginine pyrophosphate, monolysine pyrophosphate, dipotassium pyrophosphate, bisethanolamine pyrophosphate and bisammonium pyrophosphate is in the form of an oral pharmaceutical composition.

The oral pyrophosphate selected from the group consisting of monoarginine pyrophosphate, monolysine pyrophosphate, dipotassium pyrophosphate, bisethanolamine pyrophosphate and bisammonium pyrophosphate may be administered alone or in combination with other agents. The pyrophosphate selected from the group consisting of monoarginine pyrophosphate, monolysine pyrophosphate, dipotassium pyrophosphate, bisethanolamine pyrophosphate and bisammonium pyrophosphate may be administered before, after, or concurrently with such other agents or can be co-administered with other known therapies.

In an embodiment, the present invention pertains to a method for increasing plasma inorganic pyrophosphate levels in a subject in need thereof, comprising the step of administering to the subject a therapeutically effective amount of pyrophosphate selected from the group consisting of monoarginine pyrophosphate, monolysine pyrophosphate, dipotassium pyrophosphate, bisethanolamine pyrophosphate and bisammonium pyrophosphate, wherein said pyrophosphate selected from the group consisting of monoarginine pyrophosphate, monolysine pyrophosphate, dipotassium pyrophosphate, bisethanolamine pyrophosphate and bisammonium pyrophosphate is administered in oral form. Preferably the composition comprising the pyrophosphate selected from the group consisting of monoarginine pyrophosphate, monolysine pyrophosphate, dipotassium pyrophosphate, bisethanolamine pyrophosphate and bisammonium pyrophosphate is to be swallowed by the patient.

In an embodiment, the present disclosure pertains to a method for preventing and/or reducing calcification, particularly tissue calcification, particularly soft tissue calcification, and/or diseases or disorders characterized by low plasma PPi levels, in a subject in need thereof. The method is based on the surprising finding that oral pyrophosphate selected from the group consisting of monoarginine pyrophosphate, monolysine pyrophosphate, dipotassium pyrophosphate, bisethanolamine pyrophosphate and bisammonium pyrophosphate can be administered to a subject, for example a person that has low plasma PPi levels, to cause a transient increase in plasma PPi in the subject, which can inhibit calcification, particularly tissue calcification, particularly soft tissue calcification, in the subject. Since the increase in plasma PPi is transient, therapy can be tailored to inhibit undesirable or pathological tissue calcification, without inhibiting bone calcification or inducing osteomalacia.

In an embodiment, the disclosure relates to a method for reducing calcification, particularly tissue calcification (e.g. soft tissue calcification) in a subject in need thereof, by administering to the subject one or more doses of pyrophosphate selected from the group consisting of monoarginine pyrophosphate, monolysine pyrophosphate, dipotassium pyrophosphate, bisethanolamine pyrophosphate and bisammonium pyrophosphate. Each dose may contain an amount of pyrophosphate selected from the group consisting of monoarginine pyrophosphate, monolysine pyrophosphate, dipotassium pyrophosphate, bisethanolamine pyrophosphate and bisammonium pyrophosphate that is sufficient to achieve a transient increase in plasma PPi in the subject.

In an embodiment, the PPi level in the blood/plasma returns to its base level within about 24 hours, such as within 18 hours, within 12 hours, within 6 hours, or within 4 hours, after administration of the dose pyrophosphate selected from the group consisting of monoarginine pyrophosphate, monolysine pyrophosphate, dipotassium pyrophosphate, bisethanolamine pyrophosphate and bisammonium pyrophosphate. The time period between the administration of each dose may vary. For example, oral pyrophosphate selected from the group consisting of monoarginine pyrophosphate, monolysine pyrophosphate, dipotassium pyrophosphate, bisethanolamine pyrophosphate and bisammonium pyrophosphate may be administered twice a day (or one to ten times a day) a day, daily, once every two days, once every three days, once every four days, or the like.

In an embodiment, each dose of oral pyrophosphate selected from the group consisting of monoarginine pyrophosphate, monolysine pyrophosphate, dipotassium pyrophosphate, bisethanolamine pyrophosphate and bisammonium pyrophosphate that is administered to the subject contains an amount of pyrophosphate selected from the group consisting of monoarginine pyrophosphate, monolysine pyrophosphate, dipotassium pyrophosphate, bisethanolamine pyrophosphate and bisammonium pyrophosphate sufficient to achieve a transient increase in plasma PPi level, which may have a peak that is above 200%, or that is between about 40% and about 200%, such as between about 50% and 150%, between about 60% and about 125%, between about 70% and about 100%, between about 80% and about 90%, of the plasma PPi level observed in healthy subjects.

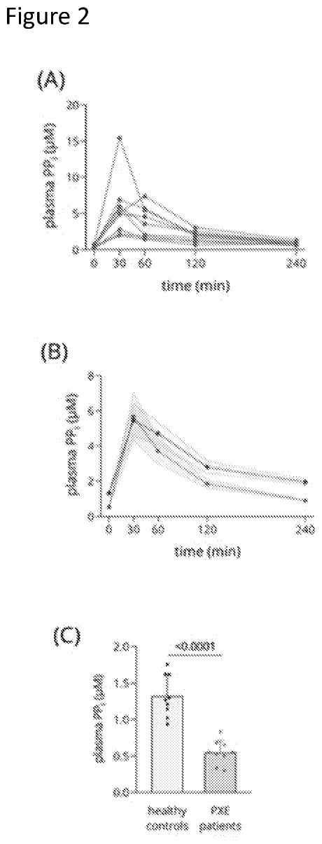

It is understood that as there may be some variation between patients with regard to plasma levels after administration of a fixed dosage of the pyrophosphate in accordance with the invention, alternatively, for each individual patient the dosage to be administered in oral form may first be determined such that peak plasma levels obtained are in a similar defined range. For example, the dosage to be administered in oral form may be determined to achieve a transient increase in plasma PPi levels in human subjects, which peak may be about 200% above the level of the plasma PPi level observed in healthy subjects, i.e. plasma levels of about 3-fold as observed in healthy subjects (see e.g. example 1 and FIG. 2C). Plasma levels in healthy subjects of PPi have been reported to be in the range of about 0.8-1.7 μM. Peak plasma levels may be selected to be at least 2 μM PPi, for example about 5 μM PPi. The peak plasma levels may be selected to be in the range of 2 μM to 20 μM PPi as observed in healthy and diseased subjects, such as shown in the examples, which were apparently well tolerated in humans. Such peak plasma levels preferably are determined 30 minutes after oral administration of pyrophosphate.

In an embodiment, the transient increase in plasma PPi level after oral administration of pyrophosphate selected from the group consisting of monoarginine pyrophosphate, monolysine pyrophosphate, dipotassium pyrophosphate, bisethanolamine pyrophosphate and bisammonium pyrophosphate is maintained for at least about 10 minutes, 15 minutes, 30 minutes, 1 hour, or the like. Further, it is preferred that the plasma PPi level returns to its base level within about 24 hours, such as within about 18 hours, 12 hours, 6 hours, 5 hours, 4 hours, 3 hours, 2 hours, 1 hour or less after administration of the dose pyrophosphate selected from the group consisting of monoarginine pyrophosphate, monolysine pyrophosphate, dipotassium pyrophosphate, bisethanolamine pyrophosphate and bisammonium pyrophosphate.

In case the subject is characterized by low plasma PPi, the low plasma PPi levels in a subject prior to treatment may be about 50% or less, 40% or less, 30% or less, 20% or less, or 10% or less, of the plasma PPi levels observed in a healthy subject.

Calcification, particularly tissue calcification is a progressive process, and individuals born with congenital deficiencies leading to low plasma PPi levels may not show tissue calcification for several years. In order to reduce or minimize calcification in such subjects, therapy should be initiated as early as possible, preferably even before tissue calcification is noticed.

In subjects with low plasma PPi levels which do not have congenital deficiencies, therapy should begin as soon as practicable; i.e. soon after the diagnosis of the conditions, such as CKD or ESRD.

According to another aspect of the invention also provided is for a compound which compound is lysine pyrophosphate 1:1 salt or monolysine pyrophosphate. It was surprisingly found that this compound is bioavailable (see the examples) and may thus be useful in the treatment of various conditions that benefit from increasing plasma levels of pyrophosphate, including such conditions as described herein. Also provided is for a composition, for example, a pharmaceutical composition, that comprises such lysine pyrophosphate 1:1 salt or monolysine pyrophosphate. Also provided is for the use of lysine pyrophosphate 1:1 salt or monolysine pyrophosphate or a composition comprising lysine pyrophosphate 1:1 salt or monolysine pyrophosphate as a medicament.

Finally, there is provided for a method for preparing lysine pyrophosphate 1:1 salt or monolysine pyrophosphate or a composition comprising lysine pyrophosphate 1:1 salt or monolysine pyrophosphate wherein the method comprises contacting 1-lysine with pyrophosphoric acid, preferably contacting the thus obtained mixture with acetone (see the examples).

It is understood that as described herein for the uses and methods in accordance with the invention, wherein monolysine pyrophosphate or monoarginine pyrophosphate is listed (or lysine or arginine pyrophosphate), these highly preferably involve mono (L)-lysine-pyrophosphate or mono (L)-arginine-pyrophosphate, respectively. Hence, also provided herein in accordance with the invention are mono (L)-lysine-pyrophosphate, a composition or a medicament comprising mono (L)-lysine-pyrophosphate, or a capsule comprising mono (L)-lysine-pyrophosphate, which are for use in the medical treatments and uses as described herein. Also provided herein in accordance with the invention are mono (L)-arginine-pyrophosphate, a composition or a medicament comprising mono (L)-arginine-pyrophosphate, or a capsule comprising mono (L)-arginine-pyrophosphate, which are for use in the medical treatments and uses as described herein.

It will be understood that all details, embodiments and preferences discussed with respect to one aspect of embodiment of the invention is likewise applicable to any other aspect or embodiment of the invention and that there is therefore not need to detail all such details, embodiments and preferences for all aspect separately.

Having now generally described the invention, the same will be more readily understood through reference to the following examples which is provided by way of illustration and is not intended to be limiting of the present invention. Further aspects and embodiments will be apparent to those skilled in the art.

### EXAMPLES

### Example 1

### Human Administration of Pyrophosphate

To reduce the ion load and the amount of sodium in PPᵢ, we first turned to the Na₂H₂P₂O₇ salt form which contains half as much sodium as the previously utilized tetrasodium (Na₄P₂O₇) variant. A case report has been published describing oral pyrophosphate treatment of a PXE patient utilizing Na₂H₂P₂O₇ dissolved in water for the prevention of reocclusion after surgery for critical limb ischaemia. To avoid the unpleasant taste we used capsulated PPᵢ salts instead of water-based solution as the form of delivery. We tested two different capsules: gelatin and cellulose. Subsequently, we determined how PPᵢ is absorbed in individuals affected by PXE compared to its absorption in non-PXE volunteers.

Human studies were conducted in accordance with the Declaration of Helsinki. The oral uptake studies involving healthy human volunteers (female and male) were conducted and all patient samples were handled in an anonymized form. The oral uptake study involving PXE patients involved mutation analysis confirming the diagnosis in PXE patients. The diagnosis of PXE was based on characteristic skin findings, supported by characteristic histopathology, and ocular and vascular involvement.

### Human Uptake and Plasma Pyrophosphate Assay

Food grade Na₄P₂O₇ (anhydrous, Code 118) and Na₂H₂P₂O₇ were purchased from ICL Food Specialist (St. Louis, Mo.) and from Fosfa (Breclav, Czech Republic). Gelatin capsules were obtained from Molar Chemicals (Halasztelek, Hungary), cellulose capsules were obtained from Capsuline (Davie, Fla.).

Volunteers and PXE patients (after overnight fasting) were involved in the studies. The duration of ingestion was less than one minute. Blood samples were collected from the vena cubiti before ingestion (0 min) and after 30, 60, 120 and 240 min into CTAD anticoagulated tubes (BD, Franklin Lakes, N.J.), and filtered through Centrisart I 300,000 MWCO filters (Sartorius, Frankfurt, Germany). Plasma PPi was subsequently assayed by the luminescent method as previously described (Jansen et al. Arterioscler Thromb Vasc Biol. 2014;34(9):1985-1989).

Briefly, plasma inorganic phosphate was measured from ultrafiltered plasma used for PPi assay since about 85 to 90% of serum phosphate is free and is ultrafilterable. We have tested that the anticoagulant mixture in the CTAD blood collection tubes does not interfere with the assay. Plasma inorganic phosphate was measured by ammonium molybdate method: 10 μl of plasma sample or calibration standard was added to a mixture of 0.3 ml reagent containing 2.5 M H₂SO₄, 1% ammonium molybdate, and 0.014% antimony potassium tartrate and 0.7 ml 20% acetic acid. For the reduction of the complex 0.15 ml of 1% ascorbic acid (freshly prepared) was added. Optical density was determined after 15 minutes at 880 nm.

### Results

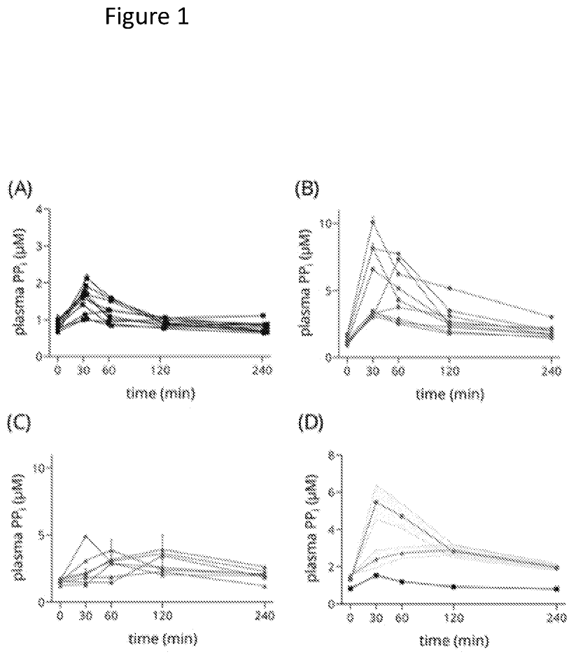

We first recorded the absorption curves of three different delivery methods: Na₄P₂O₇ dissolved in drinking water (40 mg pyrophosphate/kg), and Na₂H₂P₂O₇ loaded into gelatin or cellulose capsules (39 mg pyrophosphate/kg each), as shown in FIG. 1. There are individual differences in the extent of absorption among the healthy volunteers in the case of each delivery method, similar to our previous observations (Dedinszki et al. EMBO Mol Med. 2017;9(11):1463-1470). However, the absorption from the two different types of capsules was very different (compare panels B, C and D in FIG. 1), with gelatin capsules being more effective than cellulose. Furthermore, both capsule-Na₂H₂P₂O₇ combinations were superior when compared to the Na₄P₂O₇-water solution (compare panels a and d). The major outcome of this experiment was that the introduction of gelatin capsules comprising Na₂H₂P₂O₇ not only provided a solution for the problem of the unpleasant taste, but was shown to be most effective resulting in the highest absorption. In addition, the GI discomfort experienced by volunteers earlier with tetrasodium salt was reduced to the minimum.

The two types of capsules release their cargo in different compartments of the GI system. While gelatin dissolves within minutes of reaching the stomach, its cellulose counterpart shows minimal dissolution in the stomach, releasing the cargo in the small intestine. Without being bound by theory, the significantly more effective absorption from gelatin capsule may be, at least partly, due to the acidic environment of the stomach. At low pH the pyrophosphate anion carries less negative charge which makes passive diffusion through the organ barrier of the GI system more probable.

After establishing the Na₂H₂P₂O₇-gelatin capsule as a preferred delivery method we next determined how PPᵢ is absorbed in individuals affected by PXE compared to its absorption in non-PXE volunteers. We used the exact same experimental setup in the case of PXE patients using the same batch of Na₂H₂P₂O₇ and the same batch of gelatin capsules. FIG. 2A displays PPᵢ absorption in PXE patients, showing individual differences, while on panel B the compared absorption curves of patients versus healthy volunteers confirm very similar uptake in the two groups. The peak PPᵢ concentrations are 5.5±0.9 μM in the non-PXE group and 5.7±1.4 μM in the PXE group. Baseline concentrations, as expected, are different, 1.3±0.1 and 0.5±0.1 μM in the non-PXE and PXE group, respectively (FIG. 2C). Gastro-intestinal discomfort was not reported.

### Example 2

### Sodium-Free Pyrophosphate Forms

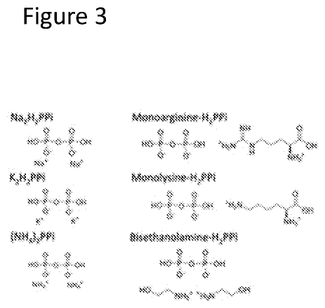

Next, we have embarked upon the development of sodium-free pyrophosphate forms to not only reduce sodium, but to avoid excessive delivery of sodium altogether. FIG. 3 shows the chemical structures of compounds synthesized for this study (K₂H₂P₂O₇, (NH₄)2P₂O₇, monoarginine-H₂P₂O₇, monolysine-H₂P₂O₇ and bisethanolamine-H₂P₂O₇) along with Na₂H₂P₂O₇.

Synthesis and Purification of the Sodium-Free Compounds

Technical grade pyrophosphoric acid from Sigma Aldrich was used for the salt forming reactions. The solid pyrophosphoric acid was covered by a layer of viscous liquid which was wiped away with a Kimwipe just prior to placing it in the reaction flask. Purity of the salts was determined by 31 P NMR Numega, SanDiego, Calif.). The only detected impurity was phosphate.

Higher polyphosphate impurities were not observed at significant levels in the salts. Karl Fisher, elemental microanalysis, and ICP-OES were performed by Robertson Microlit (Ledgewood, N.J.).

### Bis-Ethanolamine-Pyrophosphate

A flask containing pyrophosphoric acid (946 mg, 5.31 mmol) was chilled over an ice bath and treated with a pre-chilled solution of ethanolamine (648 mg, 10.6 mmol) in DI water (10 mL). The mixture was stirred for 20 min until the solid pyrophosphoric acid had dissolved and methanol was added slowly until cloudiness persisted. The flask was stored at 4° C. overnight. The resulting solid was collected in a frit by vacuum filtration, washed with cold 80% methanol/DI water and methanol, and dried overnight under high vacuum to give product as a white solid (528 mg, 99.5% purity). ³¹P NMR (D₂O): δ 10.15 (s). KF 0.10%. Anal. (C₄H₁₈N₂O₉P₂) C, H, N

### Diammonium-Pyrophosphate

A flask containing pyrophosphoric acid (1.30 g, 7.30 mmol) was chilled over an ice bath and treated with a pre-chilled solution of ammonium hydroxide (1.0 mL, 14.6 mmol) in DI water (10 mL). The mixture was stirred for 20 min until the solid pyrophosphoric acid had dissolved and methanol was added slowly until cloudiness persisted. The flask was stored at 4° C. overnight. The resulting solid was collected in a frit by vacuum filtration, washed with cold 80% methanol/DI water and methanol, and dried overnight under high vacuum to give product as a white solid (247 mg, 98.4% purity). ³¹P NMR (D₂O): δ 10.18 (s). KF 0.72%. Anal. (H₁₀N₂O₇P₂) C, H, N

Dipotassium Pyrophosphate: K₂H₂P₂O₇

A flask containing pyrophosphoric acid (1.40 g, 7.86 mmol) was chilled over an ice bath and treated with a pre-chilled solution of 1N KOH (15.72 mL, 15.72 mmol). The mixture was stirred for 20 min until the solid pyrophosphoric acid had dissolved and methanol was added slowly until cloudiness persisted. The flask was stored at 4° C. overnight. The resulting solid was collected in a frit by vacuum filtration, washed with cold 70% methanol/DI water and acetone, and dried overnight under high vacuum to give product as a white solid (445 mg, 98.6% purity). ³¹P NMR (D₂O): δ 10.10 (s). KF 2.84%. Anal. ICP-OES (H₂K₂O₇P₂O₄H₂O) K

### Mono(L)-Lysine-Pyrophosphate

A stirred solution of L-lysine (659 mg, 4.52 mmol) in deionized water (20 mL), chilled over an ice/water bath, was treated with chunks of solid pyrophosphoric acid (806 mg, 4.52 mmol, Sigma-Aldrich technical grade) which had been wiped clean of syrup. The mixture was stirred for 15-20min until the solids had dissolved and treated with 4 mL of acetone or acetone was added slowly until cloudiness persisted. The mixture was stored at 4° C. overnight. The resulting solid was filtered and washed twice with cold 20% acetone/deionized water and dried under high vacuum overnight in order to obtain lysine pyrophosphate 1:1 salt a shown below (monolysine pyrophosphate).

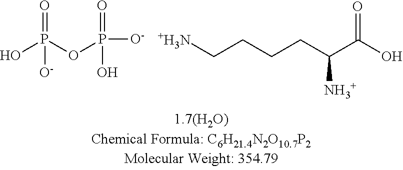 [SMILES: O=P([O-])(O)OP(=O)([O-])O ; [NH3+]CCCC[C@H]([NH3+])C(=O)O]

The product was a white solid (710 mg, 97.1% purity). ³¹P NMR (D₂O): δ 10.39 (s). KF 9.49%. Anal. (C₆H₁₈N₂O₉P₂ 1.7H₂O) C, H, N

### Mono (L)-Arginine Pyrophosphate

A flask containing pyrophosphoric acid (5.93 g, 33.3 mmol) was chilled over an ice bath and treated with a pre-chilled solution of (L)-lysine (5.8 g, 33.3 mmol) in DI water (100 mL). The mixture was stirred for 20 min until the solid pyrophosphoric acid had dissolved. Acetone (20 mL) was added, and the mixture was seeded. Acetone (5 mL) was added, and the mixture was stirred until precipitate formed. Acetone (5 mL) was added, and the flask was stored at 4° C. for 1 hr. The resulting solid was collected in a frit by vacuum filtration, washed with cold 50% acetone/DI water and acetone, and dried overnight under high vacuum to give a white solid (5.9 g). The solid was dissolved in DI water (150 mL) and chilled over an ice bath with stirring. Methanol (50 mL) was added along with a seed crystal and the mixture was stirred until precipitate formed. Methanol (50 mL) was added, and the mixture was stirred for 15 min and stored at 4° C. for 1 hr. The resulting solid was collected in a frit by vacuum filtration, washed with cold 50% methanol/DI water and methanol, and dried overnight under high vacuum to give product as a white solid (1.85 g, 99.6% purity). ³¹P NMR (D₂O): δ10.41 (s). KF 0.50%. Anal. (C₆H₁₈N₂O₉P₂) C, H, N

### Experiments of PPi Uptake in Mice

For PPi uptake studies in mice, three-month-old C57/BL6 female mice, or both males and females, were fasted for 4 hours prior to PPi administration. PPi solution was given under anesthesia via gastric gavage (the dose equivalent to 39 mg/kg PPi) in a volume of 5 μl/g. Blood was taken by cardiac puncture prior and after 10, 30 and 60 minutes (n>3 mice at each timepoint) of administration, and collected into tubes containing 50 μl CTAD (BD, Franklin Lakes, N.J.). In some experiments, also after 90 minutes blood was taken. Following filtration through Centrisart I 300,000 MWCO filters (Sartorius, Frankfurt, Germany), plasma PPi was assayed as described by (Dedinszki et al, 2017 EMBO Molecular Medicine, 2017 Nov; 9(11): 1463-1470).

### Results

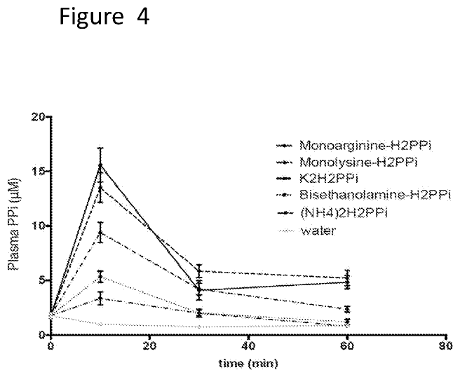

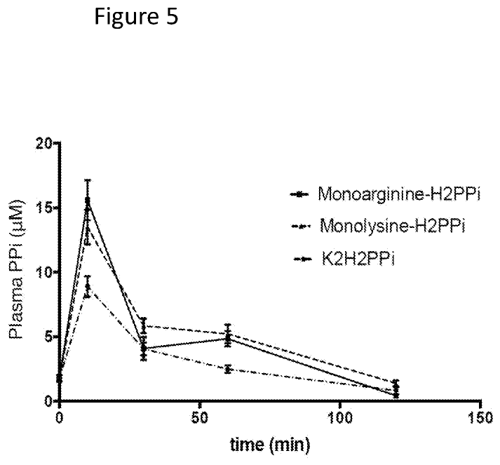

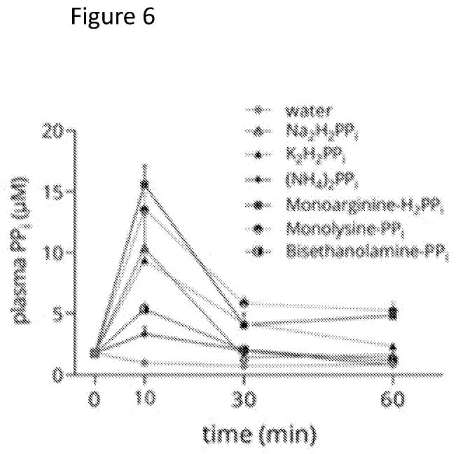

Results of PPi uptake in three-month-old C57/BL6 mice are shown in FIGS. 4-6. We have tested the absorption of the novel PPᵢ variants in mice. Oral delivery for the absorption experiments was carried out as follows: three-month-old C57/BL6 mice were fasted for 4 hours prior to PPᵢ administration. PPᵢ solution was given under anesthesia via gastric gavage (the dose equivalent to 39 mg/kg PPᵢ). Blood was taken by cardiac puncture before and after 10, and 60 minutes of delivery (n≥3 mice at each timepoint), and plasma PPᵢ was assayed as described in the Methods section. We have observed PPᵢ absorption in the case of each form, however the two amino acid derivatives, the monoarginine-H₂P₂O₇ and the monolysine-H₂P₂O₇ resulted in the highest concentration at the maximum of the absorption curve (15.6±1.5 and 13.5±1.3 μM, respectively). The potassium and sodium forms showed approximately same maximum (9.4±0.9 and 10.4±2.7 μM, respectively), while administration of the bisethanolamine and the (NH₄) 2 PPᵢ derivatives yielded only 5.3±0.5 and 3.4±0.6 μM peak plasma concentrations, respectively.

### Experiment 3: Sodium-Free Pyrophosphate in Human

Of the suitable sodium free pyrophosphates the K₂H₂P₂O₇ salt was only available in GMP quality. The K₂H₂P₂O₇ form in Good Manufacturing Practice (GMP) quality was obtained from ERAS Labo (Grenoble, France). Following our protocol utilized in the human studies (oral administration of a 39 mg PPᵢ/kg dose loaded in gelatin capsules) we investigated the properties of the potassium salt K₂H₂P₂O₇ in humans. We designed this experiment with the same healthy volunteers as described for experiment 1.

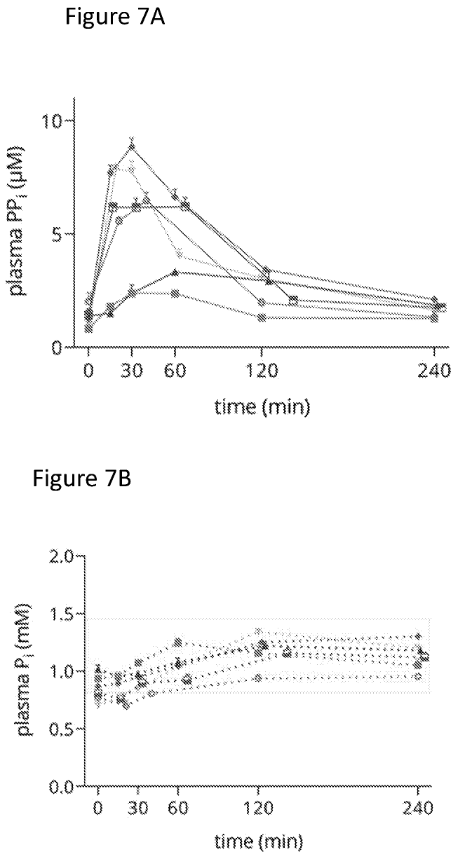

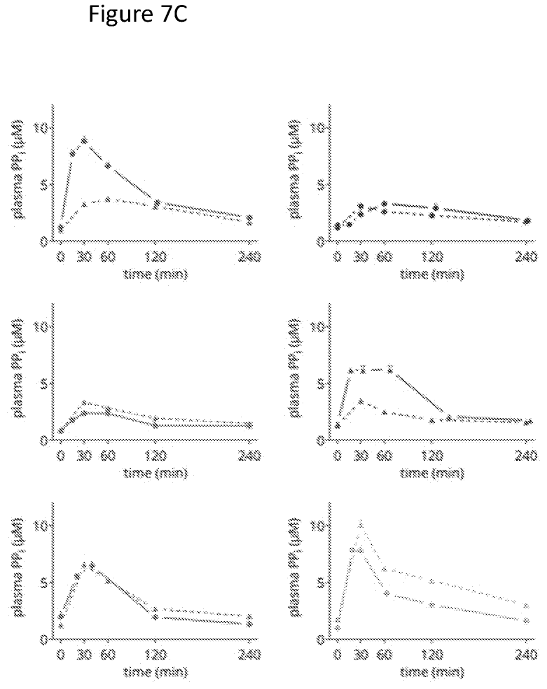

FIG. 7A summarizes the uptake curves of all volunteers involved in the study while the individual absorption plots of six healthy volunteers are presented on FIG. 7C. FIG. 7A and C panels again indicate large individual differences similar to those observed in the case of the sodium forms (see above). The individual curves demonstrate better PPᵢ uptake in two cases when K₂H₂P₂O₇was taken and approximately the same extent of absorption as with Na2H2P2O7 in the case of the other four volunteers.

Pyrophosphate is hydrolyzed in the gut to inorganic phosphate (Pᵢ) and excreted via urine and feces as indicated by a study using rats. We have determined Pᵢ concentration in the blood of volunteers during the time course of K₂H₂P₂O₇ uptake (FIG. 7B). An increase of plasma Pᵢ was detected from 0.96 to 1.34 mmol/L and with much smaller individual differences than in case of PPᵢ in plasma. These values are within the normal range of 0.81-1.45 mmol/L (Burtis et al. Fund of Clin Chem and Mol Diag—7th Edition. 7th ed. Elsevier; 2014).

Experiment 4: Sodium-free pyrophosphate inhibits ectopic calcification

Next, the capacity of the K₂H₂P₂O₇ salt in inhibiting ectopic calcification in Abcc6 mice was tested by the cryo-injury based DCC-method as described in our earlier papers.

Briefly, mice were kept under routine laboratory conditions with a 12-hour light-dark cycle and ad libitum access to water and chow. Anesthesia was carried out by intraperitoneal injection of the mixture of Zoletil (30 mg/kg, Virbac, France), Xylazine (12.5 mg/kg, Produlab Pharma, The Netherlands) and Butorphanol (3 mg/kg, Richterpharma, Austria) during all procedures. Cryo-injury was performed as described previously (Pomozi et al., J Invest Dermatol. 2017;137(3):595-602 and Dedinszki et al. EMBO Mol Med. 2017;9(11):1463-1470), with 10 mM PPi treatment provided as Na₂H₂P₂O₇ or K₂H₂P₂O₇ or no PPi added (control) to the drinking water of female Abcc6^{-/-} mice (Gorgets et al., Hum Mol Genet. 2005;14(13):1763-1773).

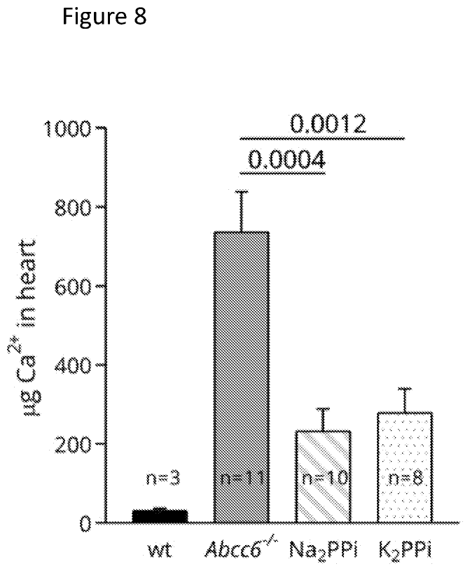

In this experiment calcification was induced in the myocardium and five days after we determined the Ca²⁺-content of the heart. As it is demonstrated in FIG. 8, this treatment does not induce mineralization in wild type mice but a massive calcification develops in the Abcc6^{-/-} animals. Both Na₂H₂P₂O₇ and K₂H₂P₂O₇, when given orally (10 mM in drinking water), reduce calcification to the same extent, indicating that the newly synthesized sodium free pyrophosophate was as effective as the sodium form. This is in line with our earlier data using the Na₄P₂O₇ form (Dedinszki et al. EMBO Mol Med. 2017;9(11):1463-1470).

### Conclusions

In previous experiments the Na₄P₂O₇ form of PPi was used and delivered dissolved in drinking water. In spite of its clear inhibitory effect, this form of pyrophosphate has disadvantages: high ion load which results in gastro-intestinal (GI) discomfort and unpleasant bitter taste. In addition, it provides for an unwanted excess of sodium intake. As outlined herein, we have now overcome these issues by providing for improved chemical forms and/or delivery methods of orally given PPi.

We have shown that a sodium-free PPᵢ derivative, K₂H₂P₂O₇, is absorbed in humans when given orally, similar to Na₂H₂P₂O₇. This overcomes the problem of the excess sodium intake. Importantly, we demonstrated that sodium-free PPi derivate K₂H₂P₂O₇ effectively inhibits calcification in Abcc^{-/-} mice. Our results also indicate that a formulation in gelatin capsules may provide for the highest uptake. These findings suggest that the K₂H₂P₂O₇-gelatin capsule formulation is a suitable candidate for human use. Furthermore, we have identified further sodium-free pyrophosphate forms (in particular monolysine- and monoarginine derivatives), which show high absorption in animal experimental models when given orally, which are regarded highly suitable for human use as well.

Individual differences in uptake of pyrophosphate both in the group of healthy volunteers and in the group of PXE patients were observed. The dose we have applied was chosen to provide at least 2.5 fold elevation of plasma pyrophosphate concentration at the peak level even in the case of the lowest absorption efficacy (see FIG. 7). It is notable that a daily transient increase of PPᵢ in the circulation was sufficient to inhibit mineralization in Abcc6^{-/-} mice. The observed individual variation in absorption may indicate personalized dosing established prior to therapy may be advantageous.

Arterial media calcification observed in PXE is also present in patients with diabetes, chronic kidney disease and as a result of ageing, which is often accompanied by macular degeneration. PXE can be considered as a model to study arterial medial calcification in cardiovascular disease, and research can also lead to novel therapeutic interventions which would be relevant not only in PXE but could also reduce calcification and related cardiovascular risk in the above mentioned populations. The intervention based on oral administration of pyrophosphate is a highly suitable approach. Our results obtained with sodium-free pyrophosphate provide for new and highly advantageous chemical and/or administration forms of pyrophosphate of use in such interventions.

Having now fully described this invention, it will be appreciated by those skilled in the art that the same can be performed within a wide range of equivalent parameters, concentrations, and conditions without departing from the spirit and scope of the invention and without undue experimentation.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art./usa.

1. Pyrophosphate selected from the group consisting of monolysine pyrophosphate, monoarginine pyrophosphate, dipotassium pyrophosphate, bisethanolamine pyrophosphate and bisammonium pyrophosphate for use as a medicament, wherein said pyrophosphate is administered in oral form.

2. Pyrophosphate selected from the group consisting of monolysine pyrophosphate, monoarginine pyrophosphate, dipotassium pyrophosphate, bisethanolamine pyrophosphate and bisammonium pyrophosphate for use in preventing and/or treating diseases or disorders characterized by calcification, particularly tissue calcification, particularly soft tissue calcification, or diseases or disorders characterized by low plasma inorganic pyrophosphate (PPi) levels, wherein said pyrophosphate is administered in oral form.

3. Pyrophosphate selected from the group consisting of monolysine pyrophosphate, monoarginine pyrophosphate, dipotassium pyrophosphate, bisethanolamine pyrophosphate and bisammonium pyrophosphate for use according to claim 1, wherein the soft tissue calcification is vascular calcification such as arterial calcification or intimal calcification.

4. Pyrophosphate selected from the group consisting of monolysine pyrophosphate, monoarginine pyrophosphate, dipotassium pyrophosphate, bisethanolamine pyrophosphate and bisammonium pyrophosphate for use according to claim 1, wherein the tissue calcification is in a subject having ENPP1 deficiency, chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type I and II, cardiovascular disorder, calciphylaxis, calciphylaxis secondary to chronic kidney disease, calcific uremic arteriolopathy or atherosclerosis.

5. Pyrophosphate selected from the group consisting of monolysine pyrophosphate, monoarginine pyrophosphate, dipotassium pyrophosphate, bisethanolamine pyrophosphate and bisammonium pyrophosphate for use according to claim 1, wherein the pyrophosphate is to be administered to a human subject.

6. Pyrophosphate selected from the group consisting of monolysine pyrophosphate, monoarginine pyrophosphate, dipotassium pyrophosphate, bisethanolamine pyrophosphate and bisammonium pyrophosphate for use according to claim 1, wherein the pyrophosphate is to be administered daily.

7. Pyrophosphate selected from the group consisting of monolysine pyrophosphate, monoarginine pyrophosphate, dipotassium pyrophosphate, bisethanolamine pyrophosphate and bisammonium pyrophosphate for use according to claim 1, wherein the daily dose of the pyrophosphate administered is 10-1000 mg per kilogram bodyweight.

8. A method for preventing and/or reducing calcification, particularly tissue calcification, particularly soft tissue, calcification, and/or diseases or disorders characterized by low plasma PPi levels, comprising the step of administering to a subject in need thereof a therapeutically effective amount of pyrophosphate selected from the group consisting of monolysine pyrophosphate, mono arginine pyrophosphate, dipotassium pyrophosphate, bisethanolamine pyrophosphate and bisammonium pyrophosphate, wherein said pyrophosphate is administered in oral form.

9. The method according to claim 8, wherein the soft tissue calcification is vascular calcification such as arterial calcification or intimal calcification.

10. The method according to claim 8, wherein the pyrophosphate selected from the group consisting of monolysine pyrophosphate, monoarginine pyrophosphate, dipotassium pyrophosphate, bisethanolamine pyrophosphate and bisammonium pyrophosphate is sufficient to achieve a transient increase in plasma PPi level in the subject.

11. The method according to claim 8, wherein the transient increase in plasma PPi level is characterized by a PPi level that is at least about 40% of the plasma PPi level in a healthy subject.

12. The method according to claim 8, wherein the transient increase in plasma PPi level is maintained for at least about 15 minutes.

13. The method according to claim 8, wherein the subject has a disease or disorder characterized by low plasma PPi levels, e.g., chronic kidney disease (CKD), end-stage renal disease (ESRD), generalized arterial calcification of infancy (GACI), hypophosphatemic rickets, heterotropic ossification of traumatized muscle, a cardiovascular disorder, calciphylaxis, calciphylaxis secondary to chronic kidney disease, calcific uremic arteriolopathy, atherosclerosis and/or pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, or diabetes mellitus type I and II.

14. The method according to claim 13, wherein the subject has GACI or PXE.

15. The method according to claim 8, wherein the daily dose pyrophosphate selected from the group consisting of monoarginine pyrophosphate, monolysine pyrophosphate, dipotassium pyrophosphate, bisethanolamine pyrophosphate and bisammonium pyrophosphate administered is 10-1000 mg per kilogram bodyweight.

16. A method for increasing plasma inorganic pyrophosphate levels in a subject in need thereof, comprising the step of administering to the subject a therapeutically effective amount of pyrophosphate selected from the group consisting of monoarginine pyrophosphate, monolysine pyrophosphate, dipotassium pyrophosphate, bisethanolamine pyrophosphate and bisammonium pyrophosphate, wherein said pyrophosphate selected from the group consisting of monoarginine pyrophosphate, monolysine pyrophosphate, dipotassium pyrophosphate, bisethanolamine pyrophosphate and bisammonium pyrophosphate is administered in oral form.

17. A pyrophosphate for use in accordance with claim 1, wherein said pyrophosphate is comprised in a capsule for release in the stomach, wherein said capsule is preferably a gelatin capsule.

18. A method in accordance with claim 16, wherein said pyrophosphate is administered in an oral form of a capsule for release in the stomach, wherein said capsule preferably is a gelatin capsule.

19. A compound which is lysine pyrophosphate 1:1 salt.

20. A compound according to claim 19 which is monolysine pyrophosphate.

21. A composition which comprises the compound as defined in claim 19.

22. A capsule comprising the compound as defined in claim 19.

23. The capsule as defined in claim 22 for use as a medicament.

24. A method for preparing the capsule of claim 22, wherein the method comprises contacting L-lysine with pyrophosphoric acid, preferably contacting the thus obtained mixture with acetone.
